(19) United States
(12) Patent Application Publication
(10) Pub. No.: US 2012/0083882 A1
(43) Pub. Date: Apr. 5, 2012

(54) SPINAL IMPLANT STRUCTURE AND METHOD FOR MANUFACTURING THE SAME

(75) Inventors: WEI- JEN SHIH, Kaohsiung County (TW); Yu-hsien Kao, Kaohsiung City (TW); Wei-Te Chen, Changhua County (TW); Yen-Nien Chen, Kaohsiung City (TW); Jin-Long Jou, Kaohsiung City (TW)

(73) Assignee: METAL INDUSTRIES RESEARCH&DEVELOPMENT CENTRE, KAOHSIUNG (TW)

(21) Appl. No.: 12/977,363

(22) Filed: Dec. 23, 2010

(30) Foreign Application Priority Data
Oct. 1, 2010 (TW) 099133492

(51) Int. Cl. A61F 2/44; B23P 17/00

(52) U.S. Cl. 623/17.11; 29/527.4

(57) ABSTRACT

A spinal implant structure includes a hollow cylinder and a biodegradable polymer membrane. The hollow cylinder is implanted in a bone damaged part of human vertebra. The biodegradable polymer membrane is formed to a part of a surface of the hollow cylinder. Thus, the biodegradable polymer membrane blocks invasion of soft tissues, and then the bone fillers integrated with vertebra are maintained without loss.

### CROSS-REFERENCE TO RELATED APPLICATION

This application claims the benefit of Taiwan Patent Application No. 099133492, filed on Oct. 1, 2010, which is hereby incorporated by reference for all purposes as if fully set forth herein.

### BACKGROUND OF THE INVENTION

1. Field of Invention

The present invention relates to a spinal implant structure and a method for manufacturing the same, and more particularly to a spinal implant structure, for being implanted in a bone damaged part of human vertebra for recovery of bone and capable of preventing invasion of soft tissues to avoid damages on a recovery course, and a method for manufacturing the spinal implant structure.

2. Related Art

In order to enable the surgery patients to guide tissue regeneration (GTR), recently, in the medical field, barrier membranes are used to block cells of soft tissues with a high growth rate, and a stable space environment is provided for bone cells (cementum, periodontal ligament, and alveolar bone) with a low growth rate, whereby the bone cells can be migrated, differentiated, and grown, so as to achieve effects of bone healing and teeth strengthening. The GTR technology may be further developed to guide bone regeneration (GBR) and applied to reconstruction of bone defects.

However, currently, the function of many spinal implants is mainly used on hollowed part or fixing the vertebra. During using, in order to accelerate bone tissue healing, an open space located inside the implant is filled with a bone filler, such as calcium phosphate, or autologous bone fragments. Therefore, the filler is easily lost due to an invasion of the soft tissues with the high growth rate or a circulation of the circulating system.

In Taiwan, R.O.C. Utility Model Patent No. M333885, a vertebral fixation plate assembly for improving a bone fusion efficiency is disclosed, the vertebral fixation plate assembly is physically connected between an upper centrum and a lower centrum of a removed part of the vertebra through a surgical implanting manner, whereby the vertebral fixation plate assembly is fixed in the front of or on the lateral side of the removed part. The vertebral fixation plate assembly includes a body plate, a plurality of bone screws, and a hollow cage. The body plate has a plurality of fixing holes/slots opened thereon and a hollow-out hole opened at a middle portion. The plurality of bone screws passes through the selected fixing holes/slots, and the hollow cage is sleeved and fixed in the hollow-out hole, and has an in-slot space. A plurality of bone fusion holes is opened in surrounding walls of the in-slot space for bone fusion and bone growth. The conventional structure is bonded to an artificial vertebra centrum stent through the hollow cage in the manner of bone fusion, so as to effectively avoid subsidence of the vertebra centrum and prevent the artificial vertebra centrum stent from slipping, and thus improving the bone fusion efficiency. However, as the conventional structure is merely applicable to fixation plate assemblies between centrums and must be locked and fixed by bone nails, the surgical procedure is complex, and the problem that the bone filler is lost due to the invasion of the soft tissues or the circulation of the circulating system still occur, the conventional structure still needs to be modified.

### SUMMARY OF THE INVENTION

Accordingly, the present invention is directed to a spinal implant structure, which is implanted in a damaged part of human vertebra, so as to provide effects of inter-vertebral fusion and recovery protection after operation.

In order to achieve the above objective, the present invention provides a spinal implant structure, which includes a hollow cylinder implanted in a bone damaged part of human vertebra, and a biodegradable polymer membrane bonded to a part of the hollow cylinder. Thus, in the spinal implant structure according to the present invention, the biodegradable polymer membrane blocks an invasion of soft tissues, and the bone fillers integrated with vertebra arc maintained without loss. Application of the GTR membrane product in the field of vertebra operations is a new concept for the existing products, such that the GTR membrane product not only develops a new market, but also reduces the waste of medical resources by accelerating recovery of patients.

### BRIEF DESCRIPTION OF THE DRAWINGS

The present invention will become more fully understood from the detailed description given herein below for illustration only, and thus are not limitative of the present invention, and wherein:

FIG. 1 is a schematic cross-sectional view of a spinal implant structure according to the present invention implanted in a bone damaged part of vertebra;

FIG. 2 is a side view of a spinal implant structure according to the present invention implanted in a bone damaged part of vertebra;

FIG. 3 is a schematic view of forming of a hollow cylinder of a spinal implant structure according to the present invention; and

FIG. 4 is a flow chart of manufacturing a spinal implant structure according to the present invention.

### DETAILED DESCRIPTION OF THE INVENTION

Hereinafter, a spinal implant structure according to a preferred embodiment of the present invention is illustrated with reference to the accompanying drawings.

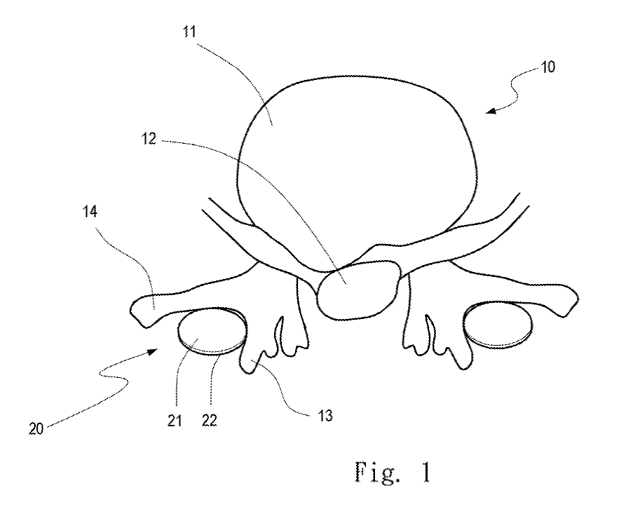

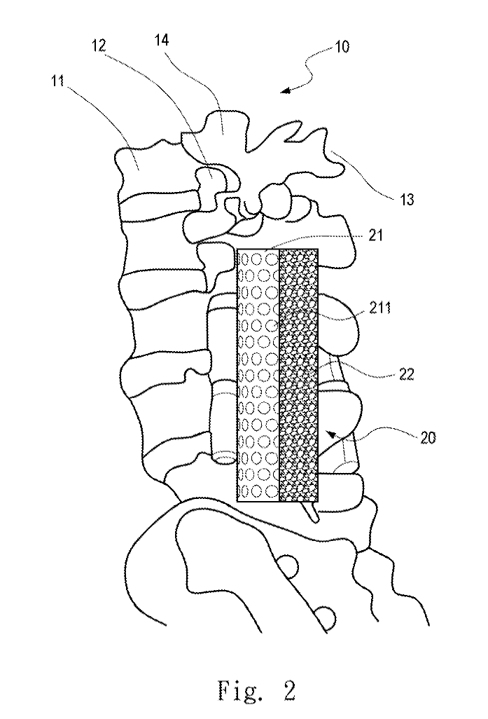

FIGS. 1 and 2 are a schematic view and a side view of a spinal implant structure respectively, the spinal implant structure implanted in a bone damaged part of vertebra according to the present invention. As shown in FIG. 1, a human vertebra 10 includes a centrum 11, a spinal nerve 12, a processus spinalis 13, and a processus transversus 14, and the like. In this embodiment, a spinal implant structure 20 is implanted between the processus spinalis 13 and the processus transversus 14, the spinal implant structure 20 includes a hollow cylinder 21 and a biodegradable polymer membrane 22 formed on a part of a surface of the hollow cylinder 21, e.g. the biodegradable polymer membrane 22 is coated on the part of a surface of the hollow cylinder 21.

As shown in FIG. 2, the hollow cylinder 21 have a plurality of holes 211 disposed therein in an array manner, and an end (e.g. a bottom) of the hollow cylinder 21 is designed to be sealed, such that a bone filler is filled from the other end (e.g. a top of the hollow cylinder 21), and herein, the bone filler is, for example, calcium phosphates, or autologous bone fragments.

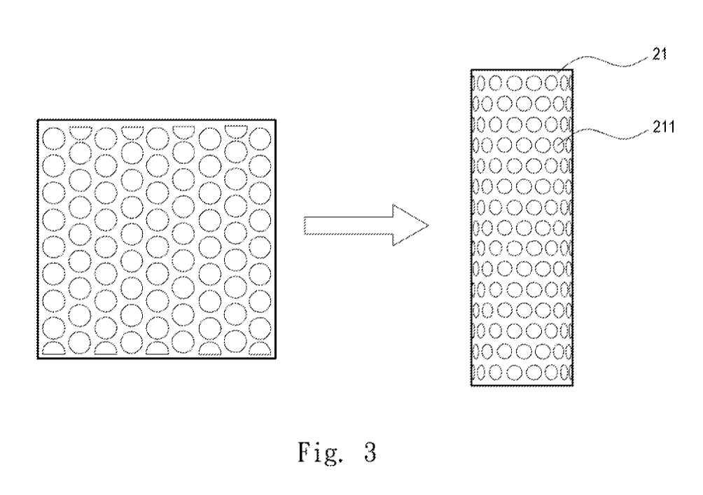

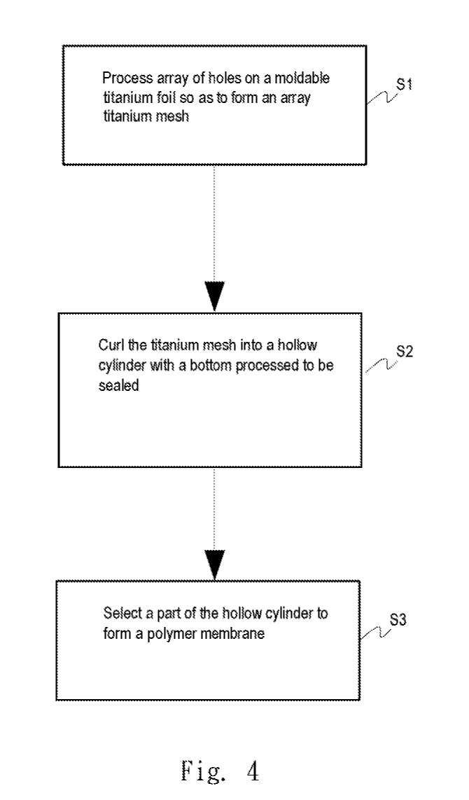

FIG. 3 is a schematic view of the hollow cylinder showing a method for manufacturing the spinal implant structure, and FIG. 4 is a flow chart showing a method for manufacturing the spinal implant structure according to the present invention. As shown in FIGS. 3 and 4, the hollow cylinder 21 may be originally a piece of titanium mesh fabricated by a moldable material, e.g. titanium foil having a thickness of about 20 μm to 200 μm (i.e. the wall thickness of the hollow cylinder 21 is 20 μm to 200 μm). In Step S1, the titanium foil is processed into a titanium mesh with a geometric array of holes which have a diameter of 1 mm to 4 mm through electrochemical or laser processing. After immersing titanium mesh in 37% hydrochloric acid for 30 minutes (min), a surface having a roughness of Ra<1.5 μm is observed. Furthermore, in this embodiment, the titanium foil may be pure titanium or titanium alloy, and the holes of the titanium mesh may be implemented to be diamond-shaped holes, circular holes, or holes of other geometric patterns.

Then, in Step S2, the titanium mesh is curled by a processing machine into the hollow cylinder 21 with the bottom processed to be sealed and the top remained open.

Next, in Step S3, a half of a surface of the hollow cylinder 21 to lean against a muscle surface is selected. The hollow cylinder 21 is first abutted in a holding mold, then a 2 to 3 wt % aqueous solution of chitosan is injected, and the hollow cylinder 21 is placed still in an oven at 40° C. and dried for about 24 hours (h), so as to form the membrane on the hollow cylinder 21. Cross-linking is performed in presence of 1N sodium hydroxide at room temperature for 3 h. Then, the hollow cylinder 21 may placed still in an oven at 40° C. and dried for about 24 h, so as to form the biodegradable polymer membrane 22 on the hollow cylinder 21. In this manner, the biodegradable polymer membrane 22 is capable of blocking the invasion of the soft tissues for 3 months to 6 months, and the part of the hollow cylinder 21 without the plated membrane is bone-integrated with the vertebra to accelerate the growth of bone tissues.

Herein, it should be noted that the biodegradable polymer membrane 22 is made of chitosan in this embodiment, but the biodegradable polymer membrane 22 may be made of collagen or gelatin except chitosan.

Finally, during an actual operation, the bone filler is filled from the top of the hollow cylinder 21, and the spinal implant structure 20 is stitched and fixed on the bone damaged part by using operation threads, so as to complete the operation.

In view of the above, in the present invention, based on the concept of GTR membrane, the spinal implant structure is manufactured as an auxiliary implant to fix the bone filler and prevent the invasion of the soft tissues, and may be used in combination with the current vertebra cage or vertebra plate. Furthermore, the spinal implant structure according to the present invention is capable of being attached to the surface of the space of the vertebra defects, and has the enough strength to endure the filling and restrict a movement space of the internal bone filler, such that the bone filler will not be lost easily, and hard tissues have a good stent space for growth. Moreover, after surface treatment of the biodegradable polymer membrane, the induced growth of the hard tissues is accelerated, meanwhile, the invasion of the soft tissues are prevented without damaging, surrounding tissues, and thus the state of the generated bone tissue is easily controlled. Additionally, when being implanted, the spinal implant structure according to the present invention is easily used and fixed without being locked with bone nails by force; and when the biodegradable polymer membrane is absorbed by the human body, the hollow cylinder has been fused well with the vertebra and needs not to be removed through another operation.

The above-mentioned is merely illustrated as an example instead of limiting the present invention. It is intended that the claims cover equivalent modifications or variations provided they fall within the spirit and the scope of the present invention.

What is claimed is:

1. A spinal implant structure, comprising:
a hollow cylinder, implanted in a bone damaged part of human vertebra, and having a sealed bottom; and
a biodegradable polymer membrane, formed on a part of a surface of the hollow cylinder.

2. The spinal implant structure according to claim 1, wherein the hollow cylinder is fabricated by a moldable material.

3. The spinal implant structure according to claim 2, wherein the moldable material of the hollow cylinder is pure titanium or titanium alloy.

4. The spinal implant structure according to claim 1, wherein a wall thickness of the hollow cylinder is 20 μm to 200 μm.

5. The spinal implant structure according to claim 1, wherein the hollow cylinder is a titanium mesh processed from a titanium foil with a geometric array of holes having a diameter of 1 mm to 4 mm.

6. The spinal implant structure according to claim 5, wherein the hole is a diamond-shaped hole or a circular hole.

7. The spinal implant structure according to claim 1, wherein the biodegradable polymer membrane is made of a chitosan, a collagen, or a gelatin.

8. A method for manufacturing a spinal implant structure, comprising:
processing a array of holes in a moldable titanium foil, so as to form a titanium mesh;
processing and curling the titanium mesh into a hollow cylinder, and sealing an end of the hollow cylinder; and
forming a polymer membrane on a part of a surface of the hollow cylinder to.

9. The method for manufacturing a spinal implant structure according to claim 8, wherein the titanium foil is made of pure titanium or titanium alloy.

10. The method for manufacturing a spinal implant structure according to claim 8, wherein a diameter of the holes is 1 mm to 4 mm.

11. The method for manufacturing a spinal implant structure according to claim 8, wherein the holes are diamond-shaped holes or circular holes.

12. The method for manufacturing a spinal implant structure according to claim 8, further comprising immersing the titanium mesh in hydrochloric acid for surface roughness treatment.

13. The method for manufacturing a spinal implant structure according to claim 8, wherein the hollow cylinder is abutted in a holding mold, and an aqueous solution of chitosan is injected to generate the polymer membrane.

14. The method for manufacturing a spinal implant structure according to claim 13, further comprising placing the hollow cylinder contacting with the aqueous solution of chitosan in an oven for being dried; then, performing cross-linking in presence of sodium hydroxide at room temperature; and placing the hollow cylinder in the oven again for being dried, so as to generate the polymer membrane.
